(19) United States
(12) Patent Application Publication
(10) Pub. No.: US 2020/0146622 A1
(43) Pub. Date: May 14, 2020

(54) SYSTEM AND METHOD FOR DETERMINING THE EFFECTIVENESS OF A COSMETIC SKIN TREATMENT

(71) Applicant: Henkel AG & Co. KGaA, Duesseldorf (DE)

(72) Inventors: ANDREAS BOCK, Neuss (DE); THOMAS WELSS, Duesseldorf (DE)

(73) Assignee: Henkel AG & Co. KGaA, Duesseldorf (DE)

(21) Appl. No.: 16/631,135

(22) PCT Filed: Jul. 2, 2018
(86) PCT No.: PCT/EP2018/067792

§ 371 Date: Jan. 14, 2020

(30) Foreign Application Priority Data
Jul. 17, 2017 (DE) 10 2017 212 176.9

(51) Int. Cl. A61B 5/00; G16H 10/20; G16H 30/40; G06T 7/00

(52) U.S. Cl. CPC A61B 5/4848; A61B 5/442; A61B 5/0077; G06Q 30/0633; G16H 10/20; G16H 30/40; G06T 7/0016; A61B 5/6898

(57) ABSTRACT

In various exemplary embodiments systems and methods for determining an efficacy of a cosmetic skin treatment are provided. In an exemplary embodiment, a system for determining an efficacy of a cosmetic skin treatment comprises: a sensor configured to determine a skin ageing information item of a user and a memory configured to store the skin ageing information item of the user determined by the sensor. A processor is configured to determine an objective skin treatment success of the user on the basis of a comparison of the skin ageing information item of the user measured by the sensor with a stored skin ageing information item stored in the memory.

### CROSS-REFERENCE TO RELATED APPLICATION

This application is a U.S. National-Stage entry under 35 U.S.C. § 371 based on International Application No. PCT/EP2018/067792, filed Jul. 2, 2018, which was published under PCT Article 21(2) and which claims priority to German Application No. 10 2017 212 176.9, filed Jul. 17, 2017, which are all hereby incorporated in their entirety by reference.

### TECHNICAL FIELD

The present disclosure relates to a system and a method for determining an efficacy of a cosmetic skin treatment.

### BACKGROUND

It is practically impossible for a consumer of cosmetic products to assess a treatment success in a standardised and objective manner following use of a cosmetic product at home. On the one hand this makes it difficult for the consumer or user to monitor individual efficacy of a cosmetic or care product, and on the other hand leads to a reduction in the motivation to perform an appropriate treatment also in the longer term.

In many areas of daily life there has been a trend, for some time now, towards personalised programs which respond to individual requirements and needs of a user, for example in the nutrition or health sector.

The treatment and care of ageing skin is an objective of many cosmetics. When skin ages a distinction is made between intrinsic and chronological skin ageing and extrinsic skin ageing. Intrinsic skin ageing reflects the normal ageing process of the body and intensifies with increasing age. Extrinsic skin ageing is triggered by environmental factors, such as smoking, UV radiation or environmental pollution and can lead to premature skin ageing or can intensify chronological skin ageing. Chronologically aged skin is often thin and transparent and has lines.

Extrinsically aged skin, in particular with chronic irradiation by ultra-violet light or sunlight, is often thickened so as to become leather-like, has deep lines, and often an irregular skin colouring. In addition, aged skin has a reduced elasticity, which contributes not only to the formation of lines, but also causes the contours to slacken, as is the case for example with sagging jowls.

The current state of aged skin can be determined by employing images which show various signs of skin ageing to varying extents, for example crow's feet, forehead wrinkles, glabella lines, sagging jowls, bags under the eyes, wrinkles above the upper lip, laughter lines, etc.

A distinction is often made between five or six levels of severity for each sign of skin ageing. Previously, the assessment was made by employing such series of images by appropriately trained experts, for example doctors trained for this purpose. A trained expert can recommend care products for example to the user on the basis of the extent of skin ageing associated with the user.

The previous systems, however, require assistance by a trained expert and are not currently available to the customer at all times and at all locations.

The skin ageing state is also perceived subjectively by the expert. It may thus be that a skin ageing state is classified differently by one trained expert than by another trained expert, for example due to different light conditions. Consequently a skin ageing state of a user might not be specified objectively.

The classification of a skin ageing state may also be performed only on the basis of defined skin ageing states that are common in the literature. Individual conditions or individual behavioural patterns of a particular user which may slow down or accelerate a skin ageing state often are not taken into consideration when determining a level of a sign of skin ageing.

When treating skin with cosmetic care products, an effect of a care product, for example an intensity of a covering by employing a cream or by employing a moisturising of a cream, or an efficacy of the care product may be heavily dependent on a skin state.

Thus, it may of great significance to determine a level of skin ageing, and this may constitute an important parameter for the user for the objective or standardised assessment of his/her skin health.

When treating ageing skin it may be desirable to be able to consult information or experience that was collected or archived during a prior treatment of ageing skin of other users with a cosmetic care product.

In the treatment of ageing skin it may be desirable to be able to include extrinsic skin ageing, which for example may be caused by environmental factors, such as smoking, ultra-violet radiation or environmental pollution, in a measurement result that provides the user for example with information regarding the current skin ageing state.

It may also be expedient to compare a skin ageing state of a user of a certain age with skin ageing states of users of the same age in order to be able to determine, for example, a treatment progress or a treatment regression. In the case of a treatment regression, another or an additional cosmetic skincare product or corresponding care recommendations may be proposed for the user, for example.

It may also be expedient to allow the user to have access to a cosmetic advice system from his/her mobile phone or his/her smartphone or his/her tablet, for example at home or on holiday.

Accordingly, it is desirable to provide a system that is able to specify an individual sign of skin ageing of a user in a standardised manner and objectively.

In addition, it is desirable to provide a system that provides a personalised cosmetic advice system which assists in slowing an ageing of the skin of a user over the course of time and can provide the user at any time and at any location with exact feedback regarding the current skin ageing state of the user.

It also is desirable to provide a system that allows the user to find cosmetic products quite selectively for the individual state of his/her ageing skin or provides assisting care advice tailored to the individual requirements of his/her skin and/or presents new innovations to the user in the field of care for ageing skin. Furthermore, other desirable features and characteristics will become apparent from the subsequent detailed description and the appended claims, taken in conjunction with the accompanying drawings and the foregoing technical field and background.

### BRIEF DESCRIPTION OF THE DRAWINGS

The present disclosure will hereinafter be described in conjunction with the following drawing figures, wherein like numerals denote like elements, and:

FIG. 1 shows a schematic depiction of a system for determining an efficacy of a cosmetic treatment in accordance with an exemplary embodiment;

FIG. 2 shows a schematic depiction of a determination of signs of skin ageing by employing a system in accordance with an exemplary embodiment;

FIG. 3 shows a schematic depiction of a system for determining an efficacy of a cosmetic treatment in accordance with an exemplary embodiment;

FIG. 4 shows a flow chart schematically illustrating a method for determining an efficacy of a cosmetic treatment in accordance with an exemplary embodiment.

Reference is made in the following detailed description to the accompanying drawings, which form part of the present application and in which specific exemplary embodiments in which the present disclosure can be carried out are shown by way of illustration.

### BRIEF SUMMARY

Systems and methods for determining the effectiveness of a cosmetic skin treatment are provided. In accordance with an exemplary embodiment, a system for determining an efficacy of a cosmetic skin treatment comprises: a sensor configured to determine a skin ageing information item of a user and a memory configured to store the skin ageing information item of the user determined by the sensor. A processor is configured to determine an objective skin treatment success of the user on the basis of a comparison of the skin ageing information item of the user measured by the sensor with a stored skin ageing information item stored in the memory.

In accordance with another exemplary embodiment, a method for determining an efficacy of a cosmetic skin treatment is provided. The method comprises capturing an image of a face of a user by using a sensor and determining a skin ageing information item of the user in the image captured by the sensor. The skin ageing information item in the image is compared to a skin ageing information item stored in a memory by using a processor and an objective skin treatment success of the user is provided on the basis of the comparison.

In a further exemplary embodiment, a system for assessing skin ageing is provided. The system comprises a camera configured to capture the image of a user and a memory storing a first sign of skin ageing. A processor is configured to detect a second sign of skin ageing from the image of the user and compare the first sign of skin ageing to the second sign of skin ageing to provide to the user an assessment of the user's skin ageing.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Systems and methods for determining the effectiveness of a cosmetic skin treatment are provided herein. Various exemplary embodiments thereof make it possible to provide an optimised cosmetic advice system which, over the course of time, can provide its data relating to a skin ageing state or skin ageing information.

Various exemplary embodiments present a system which may enable a simplification of the detection of skin ageing information and improved monitoring of the individual efficacy of a cosmetic care product tailored to a user.

In various exemplary embodiments the system may result in an increase in the motivation of a user to continue an appropriate started treatment of his/her akin with a cosmetic care product until a predetermined objective has been achieved.

In various exemplary embodiments the system may increase a satisfaction of at least one user with a care product.

In various exemplary embodiments the system may provide a deduction and/or provision of product recommendations for individual care products on the basis of at least one measured sign of skin ageing.

In various exemplary embodiments the system may provide a tracking of treatment successes by the user himself/herself.

In various exemplary embodiments the system may provide a contribution to the fact that a user of the system can enjoy independence of opening times of cosmetic advice systems, etc.

Various exemplary embodiments present a system in which a comparison of the user's own treatment progress with a treatment progress of at least one further user can be enabled.

Various exemplary embodiments present a system which can optimise a system quality on the basis of a plurality of determined signs of skin ageing of a plurality of users.

Various exemplary embodiments present a system which can refine a recommendation of care products on the basis of users already treated with care products.

Various exemplary embodiments provide a system which enables a reduction of signs of skin ageing.

Various exemplary embodiments present a system by which a user has improved control of a progress of a skin treatment and can monitor a treatment phase with a care product.

Various exemplary embodiments provide a standardised system which delivers details regarding an efficacy of a skincare product objectively.

Various exemplary embodiments provide a system which provides a possibility of improving a formulation of an individual care product by comparing the particular measurement result after use of a care product with a target state of the measured ageing skin after use of the care product.

Various exemplary embodiments provide a system in which an improvement of the skin state can be observed and archived by the user over the course of time.

Various exemplary embodiments provide a system that optimises its advice by employing at least one additional question to the particular user in relation to his/her skin state, his/her personal state of health, and further individual habits of the user.

Various exemplary embodiments provide a system which optimises an improvement of the trackability of an efficacy of a skin treatment by employing a cosmetic product or on the basis of a recommendation with regard to the use of a cosmetic skincare product.

Various exemplary embodiments provide a system which can refer to a treatment success of at least one other user who has at least one similar skin state in order to improve the treatment success of a user or at least refine a product recommendation.

Various exemplary embodiments provide a system which enables a personalised support of a user from a plurality of users.

Various exemplary embodiments provide a system which can provide a monitoring or a tracking of determined skin ageing state values by a display of measurement results determined by the system over the course of time.

Various exemplary embodiments provide a system in which a quality of the care and product recommendations can be improved by employing additional questions to a user regarding his/her skin state.

Various exemplary embodiments provide a system which can provide a user with information or feedback regarding the current skin ageing state and/or regarding a current treatment success by employing a skincare product. A reduction of a sign of skin ageing may also mean that at least one corresponding sign of skin ageing is still present, but may be made much less noticeable or visible, for example by way of a decorative cosmetic. A further objective may also lie in ensuring that at least one sign of skin ageing does not increase further.

The information may be ensured by employing the system temporally independently, in particular by the use of a mobile computer app, such that the user might not be bound by opening times for example of a doctor's practice or beauty salon.

In one exemplary embodiment a system for determining an efficacy of a cosmetic skin treatment is provided. The system may comprise: a sensor for determining a skin ageing information item of a user, a memory, wherein the memory is designed to store the skin ageing information item of the user determined by employing the sensor, and a processor, wherein the processor is designed to determine an objective skin treatment success of the particular individual user on the basis of a comparison of a skin ageing information item of the user measured by employing the sensor with a skin ageing information item stored in the memory.

In an exemplary embodiment, the sensor comprises a camera. The camera may be a camera which enables a sufficient resolution to display signs of skin ageing in an image, for example in a digital image. The camera may be provided externally or separately in accordance with various exemplary embodiments. The camera, in accordance with various exemplary embodiments, may send recorded images wirelessly or in wired fashion to a mobile device, for example to a smartphone or a tablet. In accordance with various exemplary embodiments the camera may be the camera of a smartphone, a tablet or a smart mirror. The camera, however, may be integrated in any other suitable electronic devices.

In an exemplary embodiment the system comprises a lighting source for the optimised lighting of at least a head of a user or at least one skin area when an image of the face is recorded by employing the sensor.

In an exemplary embodiment the sensor also comprises an ultra-violet camera (UV camera) for detecting an ultra-violet spectrum.

In an exemplary embodiment the camera is provided in particular for use in the field of face recognition and the recognition of signs of skin ageing in digital images. The camera may have a processor for example, which is able to automatically determine or detect faces of users of the system in an image recorded by the camera. For example, a face of a user can be associated with at least one specific stored user information item in a memory by employing a face recognition system. The camera may automatically focus the face region in particular before an image of the face of a user is recorded, and may set optimal image parameters, for example an exposure light and a white balance, a predetermined optimal skin colour, and/or colour gradients, etc. For example, an oval shape can be determined by employing a processor with use of a mathematical calculation and may make it possible to determine a possible head of a user. Furthermore, predetermined colours within the oval shape or any further conceivable shape may also be determined by employing a processor, wherein the colours for example indicate the presence of the skin pigment melanin, which occurs predominantly with any skin colour. The processor may also be designed to determine, in the previously determined shape, eyes of a user by employing digital image processing, wherein the eyes can be identified on account of their statically comparable distance and the digital image. The camera can be used to determine in computer-assisted fashion at least a face of the user with the aid of biometric data. The camera can be intended for recording frontal images and/or profile images of a head or a face or a region of the face of the user, wherein the camera can be provided to focus the face of the user regardless of a distance of the camera from the face and to record at least one image of the face.

For example, a procedure of a face recognition process may include a recording of an image of at least a head of a user. Furthermore, by employing digital image analysis, it can be checked whether a face which for example can be associated with a known face stored in a database of a memory is identifiable in the image recorded by employing a camera. In the event of a positive association of the face, the identity of the user can be assigned.

In an exemplary embodiment a method for recognising the face of a user comprises for example a finding of a face in the digital image (“face detection”), an extraction of the features of the face (“feature extraction”), and a determination of the identity (“face recognition”). The finding of a face and a digital image can be carried out by employing stored face templates. The face templates may comprise for example a plurality of different face shapes. For example, a face recognition in the digital image can be realised by employing an Elastic Bunch Graph Matching process. In the Elastic Bunch Graph Matching process, each face can be placed in a fixed grid of nodes and edges. The nodes, also referred to as fiducial points, may lie at specific points, for example at the tip of the nose or in the centre of the pupils. At each node the (colour) patterns surrounding the nodes can be stored with the aid of what are known as jets, or a set of certain wavelets. The jets may thus make the method for example insensitive to fluctuations in image brightness and an image contrast. Each of the possible jets can be associated with a fiducial point and stored in a data structure, for example in a memory. In this method a face can be detected by forming an average of the jets at a node and by comparing same with the stored image. The image is normalised in accordance with the grid. The jets of the determined fiducial points can then be calculated and stored in a memory. The produced jets may lastly be compared with the model graphs of the database so as to be able to assign the face of the user.

It should be noted that other face recognition methods and skin ageing detection methods can also be provided in other exemplary embodiments.

In an exemplary embodiment the system also determines, by employing the face recognition process, a gender of a user, an age of a user, a facial expression of a user, a detection of a head posture of a user, a detection of a viewing direction of a user and/or a detection of a blinking of a user, etc.

In an exemplary embodiment the system assists the user, for example graphically or by a voice output, as his/her face is being detected or as skin ageing information in his/her face is being detected by employing the camera. In various exemplary embodiments the system gives instructions for a position at which the user should position his/her head or his/her face in front of the camera for effective recording of signs of skin ageing in his/her face. For example, the system can thus give instructions that the user should position himself/herself frontally with his/her face in front of the camera and/or should be positioned at a predetermined angle to the side in front of the camera. The system may also give the user instructions, for example, to step closer to the camera, in order to enable an increased recording of at least one sign of skin ageing. The system may also request that the user position certain face regions in front of the camera, for example a region of the forehead for detection of forehead wrinkles or a region to the side of an eye for the detection of crow's feet. The system may also give the user an indication of how many images of the user need to be recorded for efficient measurement of the skin ageing of his/her face.

In an exemplary embodiment a method for determining signs of skin ageing in an image, an image region, a face region or a partial face region comprises, for example, a finding of a sign of skin ageing in the digital image, an extraction of the features of the sign of skin ageing, for example outlines or contours of lines, and a determination or a comparison of the extracted sign of skin ageing with a corresponding sign of skin ageing stored in a memory.

In an exemplary embodiment the system also comprises a display device. The display device may be, for example, a screen of a television, a screen or display, or a touch-sensitive screen (“touchscreen”) of a mobile phone, a smartphone, tablet or a smart mirror. However, further suitable display devices are possible according to various exemplary embodiments.

In an exemplary embodiment the sensor comprises a processor for face recognition or for recognition of signs of skin ageing, or uses an externally positioned processor for this purpose.

In an exemplary embodiment the memory comprises a database, server and/or a cloud. The memory may comprise, for example, a data record of a sign of skin ageing, a data record comprising a series of images of different levels of severity of signs of skin ageing, information based on an additional behavioural pattern of a user, information regarding a care product or a care recommendation, a target skin ageing state and/or archived or stored prior signs of skin ageing of at least one user.

In an exemplary embodiment the sensor comprises a camera.

In an exemplary embodiment the sensor is integrated in a smartphone, in a tablet or in a smart mirror and/or the memory is integrated in the smartphone, in the tablet, in the smart mirror or in an external memory device, and/or the processor is integrated in the smartphone, in the tablet or in the smart mirror.

In an exemplary embodiment the memory is provided as an external memory device.

In an exemplary embodiment the memory also comprises a stored series of images of the sign of skin ageing, and the series of images may comprise various levels of severity of the sign of skin ageing.

In exemplary embodiment a sign of skin ageing information item, such as information regarding forehead lines or worry lines, anger lines or concentration lines or glabella lines or frown lines or rage lines, bunny lines or “witch” lines, eye wrinkles or laugh lines or crow's feet, nasolabial folds, wrinkles above the upper lip or expression lines, oral commissure wrinkles, mentolabial lines, or sorrow lines or marionette lines, neck lines, sagging jowls, bags under the eyes, etc., is stored in the memory, for example. For example, different levels of severity can be assigned to different data storage cells in the memory.

In an exemplary embodiment the levels of severity of a sign of skin ageing can be divided into, for example, five, six, etc. levels of severity. The levels of severity may be categorised for example by employing the terms “mild”,

“normal” or “severe” or similarly. However, in accordance with various exemplary embodiments any number of levels of severity is possible. The number of levels of severity may also be adapted subsequently, for example minimised or refined.

Furthermore, a cosmetic care product and/or a cosmetic care recommendation may be associated in the memory with a level of severity of a sign of skin ageing and/or may be retrievable in the memory and/or readable from the memory.

In an exemplary embodiment the system also is designed to take into consideration a further individual information item, based on a dietary habit of the user, a state of health of the user, and/or an individual behavioural pattern of the user, when determining an objective skin treatment success of the user.

The individual information item based on a dietary habit may comprise, for example, information regarding the consumption of unhealthy foods which may influence the appearance of skin ageing.

The individual information item based on a state of health of the user may comprise, for example, an existing illness, sunburn caused by staying out too long in the sun or frequent visits to a tanning salon, etc.

The individual information item based on an individual behavioural pattern of the user may comprise, for example, smoking, drinking alcohol, frequent visits to a tanning salon, etc.

In an exemplary embodiment the processor is designed to store the skin ageing information item for a user of a plurality of users of the system in the memory over the course of time.

The processor may consequently archive regularly recorded images of signs of skin ageing chronologically for each user. A history data record can be provided, for example graphically, to the user from the data archived continuously in the memory over a course of treatment of an ageing skin.

In an exemplary embodiment the system provides a graphical presentation of severities of various signs of skin ageing and/or a cumulated total value following a detection of a sign of skin ageing and a comparison of a skin ageing information item stored in the memory with a sign of skin ageing detected by employing the camera. The system may provide, as a cumulated total value, an addition of a state of each different sign of skin ageing determined in a face, for example. In accordance with an exemplary embodiment, the system provides a percentage for each determined sign of skin ageing on the basis of the determined signs of skin ageing. The system may also provide a ratio of the skin ageing state measured by employing the sensor to a particular target skin ageing state. For example, it may thus be displayed graphically by employing the system that a regression of the skin ageing state to a target value or a reference value has been reached, or a regression of the skin ageing state to a target value has not been reached. Furthermore, a comparison of the currently measured skin ageing state in comparison to a skin ageing state at the start of the skin treatment by employing a cosmetic care product may be provided by employing the system.

In an exemplary embodiment the processor compares an image recorded by employing the sensor with an image of a plurality of images stored in a series of images and/or compares the image recorded by employing the sensor with at least one earlier skin ageing information item of the user stored in the memory.

In an exemplary embodiment a progress of the treatment of the ageing skin of the user is consequently determined. Feedback may be provided to the user regarding the treatment process of his/her ageing skin up to that point.

In an exemplary embodiment the system comprises a computer application (computer app).

Herein, a computer application or a computer app may comprise an application software for a mobile device, for example a smart phone or tablet. The computer app may be installed for example on the smartphone, the tablet or on a further mobile device. The computer app may be provided in platform-dependent form, for example an android app, a Windows app, or an iOS app, or in platform-independent form, for example in the form of a web app. The computer app may be designed for example to display information to the user graphically regarding his/her skin ageing state and measurement results over the course of time so as to thus monitor or track the skin ageing state. The computer app may also be intended to provide the user with individual care and product recommendations. The computer app may also ask the user of the computer app questions regarding his/her skin condition, his/her dietary habits, his/her general state of health and further behavioural patterns, which the user can answer by making inputs into the computer app. The computer app may use the additional answers of the user to optimise the quality of provided care products or care recommendations. The computer app may also be able to process the inputs made by a user. The computer app for this purpose may access a memory or a literature database comprising information regarding skin ageing information, for example.

In an exemplary embodiment the computer app is provided on/in a smart mirror (“intelligent mirror”). A smart mirror may be an intelligent mirror or a mirror signal hybrid display. In this case, advantages of a mirror can be combined with those of a digital display. The smart mirror may be used on the one hand as a conventional mirror. On the other hand, the smart mirror may be used for example as a visual presentation screen or an advertising board. The smart mirror may display visually clear real-time mirror images of a user and supplementary content in addition to the mirror image or overlapping with the mirror-image, for example by employing a high degree of reflection. The smart mirror for example may display historical images of skin ageing states over the course of time. A smart mirror or a plurality of smart mirrors may be positioned for example at a point of sales. The term “point of sales” (PoS) may be used herein to refer to a location of a range of products where the user may come into contact purposefully with the system. These locations may be for example a beauty salon, a pharmacy, a perfume shop, etc. By employing the smart mirror, provided skincare products or skincare recommendations can be provided digitally on the smart mirror. The smart mirror may be operated by a user for example by employing a touchscreen.

Furthermore, the smart mirror may have a wireless connection, for example a WLAN, NFC, Thread or Bluetooth connection, so as to exchange data with a sensor, or with a mobile device, for example a smartphone or tablet, a processor or a memory. The smart mirror may be controlled for example by employing a smartphone or tablet. The smart mirror for example may have a full-image mode, in which the majority of the mirror area can be covered with digital information. The smart mirror may also be provided in the form of a mobile device.

In an exemplary embodiment the computer app records an image of a face region of a user by employing the sensor, to store the recorded image in a memory, to compare the recorded image with a skin ageing information item of the user stored in the memory and/or skin ageing information of a further user stored in the memory and/or with stored series of images or signs of skin ageing, and to output a treatment success on a display device.

In an exemplary embodiments the system provides a skincare product tailored individually to the user and/or a skin recommendation tailored individually to the user on the basis of the comparison performed by employing the processor.

In an exemplary embodiment a skin cream associated with a certain skin ageing state for example is determined by employing the processor and/or is provided graphically for example by employing a display device described herein. Furthermore, on the basis of the determined skin ageing state, a care recommendation associated with the skin ageing state can be provided graphically on a display device described herein.

In an exemplary embodiment the system determines an individual efficacy of a skin treatment of the user over the course of time on the basis of the care product provided to the user.

In an exemplary embodiment an individual efficacy of a skin treatment by employing a cosmetic care product on the basis of a care product provided to the user for example is provided in such a way that a plurality of the recorded images of the ageing skin of a user stored in the memory can be displayed adjacently, for example next to one another, over the course of time on a display device described herein.

In this regard, a motivation to continue a started treatment of ageing skin may be increased. A likelihood of stopping an ageing skin treatment may thus be minimised.

In an exemplary embodiment a method for determining an efficacy of a cosmetic skin treatment is provided. The method comprises: capturing an image of a face of a user by employing a sensor, determining a skin ageing information item of the user in the image captured by employing the sensor, comparing the determined skin ageing information item in the image with a skin ageing information item stored in a memory by employing a processor, and providing an objective skin treatment success of the particular individual user on the basis of the comparison.

In an exemplary embodiment the skin ageing information item or a skin ageing state or a sign of skin ageing is determined by employing the method described herein.

In an exemplary embodiment the method comprises the fact that the skin ageing information stored in the memory comprises a series of images of degrees of severity of the sign of skin ageing.

In an exemplary embodiment the comparison comprises a determination of a difference between the skin ageing information item stored in the memory and a skin ageing information item measured later by the sensor.

In an exemplary embodiment the comparison comprises a determination of a difference between the skin ageing information item stored in the memory and a target skin ageing information item stored in the memory.

In an exemplary embodiment the method comprises providing care recommendations and/or skincare products tailored individually to the skin ageing information item on the basis of the skin ageing information item measured by employing the sensor and/or a target skin ageing information item.

Exemplary embodiments of the systems and methods contemplated herein are shown in the figures and will be explained in greater detail hereinafter.

In this regard, directional terms, such as “top”, “bottom”, “in front of”, “behind”, “front”, “rear”, etc. will be used with reference to the orientation of the described figure(s). Since components of exemplary embodiments can be positioned in a number of different orientations, the directional terms are used by way of illustration and are in no way limiting. It goes without saying that other exemplary embodiments can be used and structural or logical changes can be made without departing from the scope of protection of the present disclosure.

It goes without saying that the features of the various exemplary embodiments described herein can be combined with one another unless specifically stated otherwise.

The following detailed description therefore is not to be interpreted as limiting, and the scope of protection of the systems and methods contemplated herein is defined by the accompanying claims.

A digital image can be understood herein to mean a data packet which can be displayed by a data processing system as a two-dimensional (planar) arrangement of image dots, for example in a coordinate system which has an x-axis and a y-axis, wherein each image dot is associated with at least one colour information, which for example can be displayed as the colour of a pixel of a monitor or a printed dot of a printed image. The digital image can be for example a photo recorded using a digital camera or an individual image of a video sequence recorded using a digital camera.

The terms “skin region” and “skin area” are used herein synonymously. The skin region may comprise a plurality of image dots of a digital image, which depict the skin, preferably the skin of a face of a user, and which can form a continuous area or an area existing of a plurality of individual areas. A plane in which the hair region can be arranged can be determined for example by the x-axis and the y-axis of the digital image.

A “colour” can be understood herein to mean the combination of a shade (i.e. of a spectral colour impression, also referred to as hue, which can be understood to mean that which is considered to be the “actual colour”), a colour intensity (i.e. how intense the colour looks, for example compared with a neutral grey, which is also referred to as saturation, colour saturation, colourfulness, chromaticity or colour depth) and a brightness (i.e. how light or dark the colour looks).

In various exemplary embodiments the colour information includes, for example, a parameterisation in a known colour space, for example in a L*a*b colour space (wherein L* specifies the brightness of a colour, a* specifies the green and red component of the colour, and b* specifies the blue and yellow component of the colour), in an RGB colour space by colour components in red, green and blue, in a CMYK colour space by colour components in cyan, magenta, yellow and black, or in any other colour space.

The term “colour shade” may be understood herein to mean a colour value or the spectral colour impression of a colour, regardless of how this can be parameterised, for example as a point in a two-dimensional colour space (for example a*b* of the L*a*b* system) or a ratio of colour components (such as in the RGB colour space or in the CMYK colour space).

In various exemplary embodiments a colour space from which the colour information originates (skin colour information and image colour information) is such that a determined or presented colour is independent of a medium by which the colour the colour is determined or presented (for example screen, printer, scanner, human eye, etc.). The colour space may be, for example, an L*a*b* colour space, and the colour information may be a colour shade parameterised by employing a* and b*, for example.

Where reference is made herein to a smartphone, this shall be understood to be representative for all similar types of portable data processing devices, i.e. smartphones, tablets, laptops, etc., unless otherwise evident from the context. The same is true for smartphone cameras, etc.

Signs of skin ageing in a human may comprise or may be, for example, forehead lines or worry lines, anger lines or concentration lines or glabella lines or frown lines or rage lines, bunny lines or “witch” lines, eye wrinkles or laugh lines or crow's feet, nasolabial folds, wrinkles above the upper lip or expression lines, oral commissure wrinkles, mentolabial lines, or sorrow lines or marionette lines, neck lines, sagging jowls, bags under the eyes, etc. The above list is not, however, exhaustive. Further signs of skin ageing are conceivable.

The term “forehead lines or worry lines” means dynamic lines which form predominantly by mimic contraction of the face muscles, for example when worried or angry. For example the horizontal lines are formed on the forehead by dry skin.

The term “anger lines or concentration lines or glabella lines or frown lines or rage lines” means mimic lines which can form predominantly when the mimic muscles above the nasal root are tensed. Anger lines may occur horizontally, vertically, or diagonally.

The term “bunny lines or ‘witch’ lines” means lines which are found to the side of the bridge of the nose.

The term “eye wrinkles or laugh lines or crow's feet” means folds in the eye region that give the appearance of actual crow's feet due to their radial structure. Crow's feet are visible in particular in the case of dry skin, since the dry skin usually has only a low resilience.

The term “nasolabial folds” means lines which are found to the side of the bridge of the nose. These types of lines are formed by wrinkling the nose or when laughing.

The term “wrinkles above the upper lip or expression lines” means lines that are usually vertical and are found at the upper lip. These types of lines may be formed for example by the natural ageing process, but also may be caused by years of smoking. These types of folds also occur often as a result of skin that is too dry.

The term “oral commissure wrinkles” means lines that run transverse to the chin, which may lead to an uneven look of the chin. These lines are formed by a mimic contraction of the face muscles.

“Mentolabial lines, or sorrow lines or marionette lines” refer to lines that run from the corner of the mouth towards the chin.

The term “neck lines” means lines in the neck region. They are formed for example by the natural skin ageing process, ever-thinning skin, and a reduced level of fatty tissue beneath the skin, and by muscle contractions. If the lines run vertically, this is called “turkey neck”. Vertically running lines in the side region may also be accompanied by transversely running lines.

The term “bags under the eyes” refers to saggy skin folds below the eyes.

The term “sagging jowls” means saggy cheeks hanging downwardly.

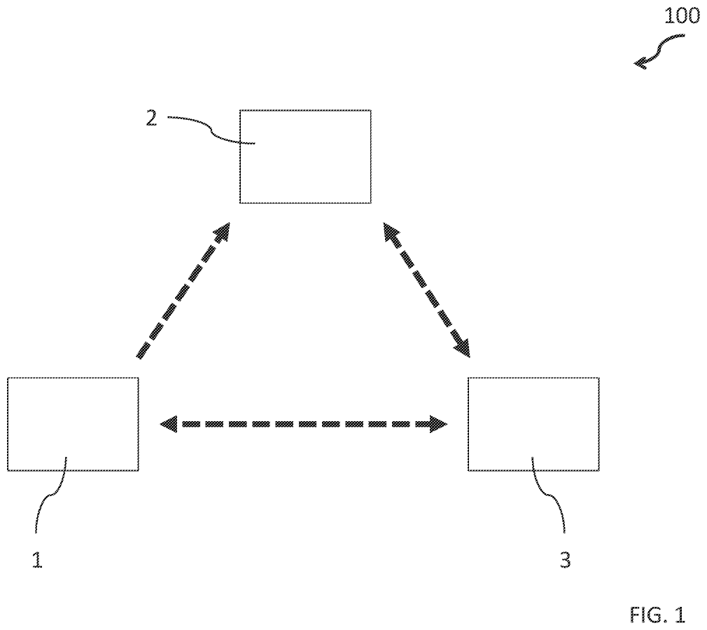

FIG. 1 shows a schematic depiction of a system 100 for determining an efficacy of a cosmetic treatment in accordance with an exemplary embodiment.

The system 100 for determining an efficacy of a cosmetic treatment comprises a sensor 1 in accordance with an exemplary embodiment. The sensor 1 may comprise at least one digital camera. The digital image captured by employing the digital camera may provide accurate skin colour tones, contours, skin surface differences in the form of wrinkles or lines, etc. for a later analysis of the digital image by employing a processor 3.

The system 100 for determining an efficacy of a cosmetic treatment further comprises a memory 2 in accordance with an exemplary embodiment. The memory 2 may be a large-scale data memory, for example a data server, a cloud server, a constant information miner server (KNIME server) or a big-data KNIME server. Systematic applications of statistical methods to large data volumes can be implemented by the use of a KNIME server. A simple and rapid linking to one another of modules for the data preprocessing of customer data can be implemented by employing a graphical user interface with use of a KNIME server.

The system 100 for determining an efficacy of a cosmetic treatment also comprises the processor 3 in accordance with an exemplary embodiment. The processor 3 may be used as an arithmetic unit in order to write information or data into the memory 2 or read information or data from the memory 2 or calculate an information item from the information item provided from the memory 2 and the at least one image recorded by employing the sensor 1.

The sensor 1 may be connected to the memory 2 by employing a wireless connection or the connection between the sensor 1 and the memory 2 may be provided in a wired manner The processor 3 may be connected to the memory 2 by employing a wireless connection. The connection between the memory 2 and the processor 3 also may be provided in a wired manner In an exemplary embodiment the processor 3 is coupled to the sensor 1 for data exchange. In an exemplary embodiment the processor 3 is provided in the sensor 1.

The system 100 for determining an efficacy of a cosmetic treatment, in accordance with an exemplary embodiment, provides a processor 3 that can receive information or data wirelessly from the sensor 1 and can forward these data for example to the memory 2 and can store them in the memory 2. In accordance with an exemplary embodiment the processor 3 is provided in the memory 2. A wireless connection between the sensor 1, the memory 2 and/or the processor 3 may be provided for example by employing a WLAN or Bluetooth connection. However, any suitable radio link can be provided.

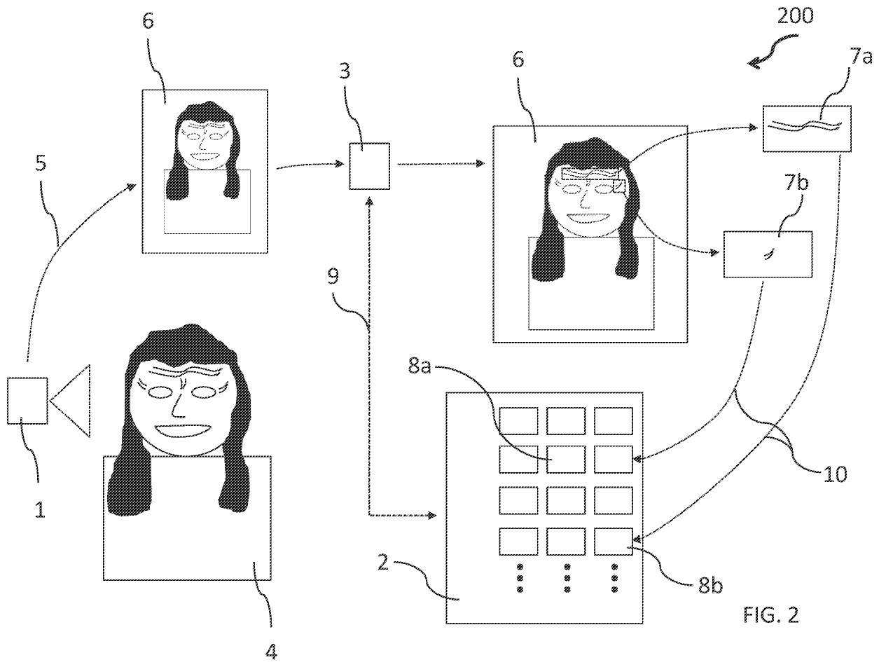

FIG. 2 shows a schematic depiction of a system 200 for determining an efficacy of a cosmetic treatment in accordance with an exemplary embodiment.

The system 200 for determining an efficacy of a cosmetic treatment comprises a sensor 1 as a camera in accordance with an exemplary embodiment. A user 4 may position his/her head or his/her face in front of the camera in such a way that an image 6 of the user 4 can be recorded by employing the camera.

In an exemplary embodiment the system 200 assists the user 4 when recording his/her face or when recording a region of his/her face. The system 200 provides the user 4 for example with graphical information relating to the way in which the user 4 should orientate his/her face in relation to the camera, for example relating to a frontal orientation of the face in front of the camera or a side arrangement of the face in front of the camera. In an exemplary embodiment the camera records the image 6 of the face of the user 4. In another exemplary embodiment the camera records a plurality of images 6.

In an exemplary embodiment the camera 1 comprises the processor 3, which, in the image 6, in particular in a digital image, determines a face region or a face area of a head imaged in the image 6.

In an exemplary embodiment the camera provides the image 6 in the form of digital image values to a processor 3 by employing a wireless connection 5. In another exemplary embodiment the camera provides the image 6 to the processor 3 by employing a wired connection 5. The processor 3 may determine a sign of skin ageing 7a, for example a forehead line, or a sign of skin ageing 7b, for example a crow's foot, on the basis of the digital image 6 by employing a method of digital image processing.

In an exemplary embodiment the system 200 is provided by employing a computer application (computer app). The computer app may thus be installed on a mobile device, for example on a smartphone, on a tablet, or on a smart mirror (“intelligent mirror”). The sensor 1 and the processor 3 may be integrated in the mobile device. The computer app may provide or display information to the user regarding a skin ageing state or a treatment progress graphically or in text form by employing a display device. The computer app may receive a user input via the display device, for example by a touchscreen, as information and/or may forward it to the memory 2. In other words, the user may record an image of his/her face from a suitable position for example by employing his/her smart phone, on which the computer app is installed. The computer app may store the measured information item in the memory 2. The user may be provided with information regarding a skin ageing state of his/her skin via the screen of his/her smartphone. The system 200 may ask the user 4 a further question regarding an individual behavioural pattern of the user by displaying the question on the screen of the smartphone. The user 4 may input an answer to the question, for example by inputting information via a touchscreen of his/her smart phone. The information input by the user 4 may be archived by the processor 3 in the memory 2 and/or incorporated in the information provided to the user 4.

In an exemplary embodiment a connection to an external large-scale server or to a cloud, which stores the information or the data for a plurality of users and can allow access to the information from outside by the computer app, may be established by employing the computer app. In an exemplary embodiment the memory 2 comprises the large-scale server.

In an exemplary embodiment a face region of a user in the digital image and/or a sign of skin ageing in the digital image is determined by employing a software-side image analysis process. In order to enable the processing of large images it may be advisable to divide the digital image into individual sub-images (image segments). Individual image parts may be associated explicitly with an object, for example the face region (for example forehead, nose, eye, mouth, etc.) of the user or a skin ageing state, without the rest of the image being processed further. This may result in a large data reduction for the further calculation. An image may be segmented in accordance with very different methods, for example by a segmentation in respect of a colour or in respect of a discovery of regions within a closed contour.

For example, regions in the digital image recorded using a camera can be determined by employing the clustering method. In the case of clustering, two centres in the digital image are firstly selected freely; one centre may be a centre point of class 1, the other centre may be a centre point of class 2. Adjacent areas around the centres having identical properties are then searched for and added to the corresponding class. Areas in the digital image are formed. Depending on the requirements, further centres may be added, so that continuous areas with identical properties ultimately form a class.

For example, regions in the digital image can be determined by employing the method of amplitude projection. If certain features of an image are known, for example a shape or a colour gradient of a sign of skin ageing, a relatively simple method of segmentation may thus often be provided. If the digital image for example contains just one region that is notable by colour gradient and that is to be found, the amplitude projection method thus lends itself. A value is calculated per row and column of a digital image recorded by employing the camera and specifies the mean value of the grey values of this one row or column. For example, if it is known that the searched-for region in the digital image is the only dark region, for example caused by at least one line, the digital image may thus be digitally trimmed by choosing all rows or columns in which the value exceeds a certain threshold value, and the rest can be masked out.

A contour tracking in the digital image in order to separate regions in the digital image by employing polygons may be helpful. In this method, instead of associating each pixel of a digital image area with its object, an encircling is performed on the basis of a coordinate list. During the contour tracking, in particular the steps of finding contours, linking contours and approximating contours may be performed. When finding contours, what is known as a bug follower is used. A bug follower moves by employing an algorithm over the pixels of the digital image and examines the contour until it has reached the starting point again.

A contour linking may then be used in particular if lines in the digital image are interrupted by external influences. It is identified with local or global operators whether, for example, a line can be continued or whether it can bypass noise or an error. The contour may then be approximated by a function, which can also be referred to as contour approximation. As a result of this further step, subsequent evaluation is also possible, because functions can be analysed or compared much better as point sequences.

In the case of an image analysis of the digital image, the processor 3 may be able to detect the position or the coordinates of a sign of skin ageing in the digital image 6 and on this basis for example can determine where the sign of skin ageing is arranged in the digital image 6.

In particular, the segmentation and the feature extraction may also be applied. In particular the methods of thresholding and of region growing, which are employed for the segmentation of digital images, can be used. In the case of detection extraction from digital images, what is known as a Canny edge detector and/or what is known as the Harris Corner Detector can be used, for example. In the segmentation of images there are three large groups of segmentation methods: threshold-based, edge-based and region-based segmentation. For example, in the case of a threshold-based segmentation, also referred to as thresholding, it is possible to work with one or more statistical or dynamic threshold values. Thresholding is the application of a threshold value to a grey image. A non-linear function converts the recorded image into a binary digital image, in which each pixel is coloured either white or black depending on whether the examined pixel value lies above or below a defined limit value.

The method of region growing may also be used to identify identical image regions. Starting from a start pixel, adjacent pixels are checked to ascertain whether they have a similarity in order to be added to the region. If this is not the case, they are rejected as lying outside. In order to obtain sensible regions, it is necessary to define seed points. The region-growing algorithm presupposes that the object to be searched for in the digital image differs sufficiently from the background, for example a reference colour on the calibration means. Consequently it may be necessary to search for selected features in the digital images which correlate to objects. To this end edge detectors are suitable in particular, since edges are distinctive image features. An edge usually forms the termination of an object, for example an edge of a reference colour or an edge of a marker. An edge, however, may also occur in other contexts. Furthermore, an edge may be exemplified by an intensity difference, for example a colour intensity difference of a reference colour as compared to its surroundings.

Generally, edge detectors work in three fundamental steps: A mask is applied to the digital image 6 in order to produce intensity gradients. Significant gradients are selected by employing thresholding (threshold value monitoring) over the gradient image.

The calculated data are then related to edges, for example to the edges of a sign of skin ageing, by employing operators and algorithms

For example, a position of an edge of a non-natural colour gradient in a face of the user can be determined in the digital image. In an exemplary embodiment a position of a sign of skin ageing is determined in the digital image 6.

Besides the edge detectors, however, the corner detectors may also be used. Two edges end in a corner, which in turn represents an object termination, for example a termination of a line, and the coordinates of which in the digital image can be detected. Corners may be determined in the digital image 6 for example by employing gradients or with the aid of morphology. One objective of the morphological image processing on the one hand may be a new image, which highlights the relevant information. A further objective may be a list which is filled with measurands determined from the digital image 6. For example, a position of a corner of a non-natural colour gradient in the face of the user can be determined in the digital image.

In an exemplary embodiment, objects such as a sign of skin ageing or a region which has a characteristic skin colour is determined in the digital image 6 by employing template matching. Template matching belongs to model-based segmentation. In the case of template matching, by employing a complete search, it is decided how well a predefined model (template) matches a specific pixel range (match). The model describes, as precisely as possible, the object to be searched for and its embedding in the surroundings. The object template is limited at a pixel distance from a bounding box. A larger surrounding environment than necessary would merely increase the error rate. The template should, as far as possible, have the same size and orientation as the object to be determined in the digital image 6. For example, a template of a skin ageing state from a plurality of skin ageing states may be created and known to the algorithm that runs on the processor 3. This template may be determined at least once in the digital image 6 by the processor 3, and the position of the template in the digital image 6 may also be determined. If, for example, the template, for example of a skin ageing state, in the digital image 6 and the orientation of the template in the digital image 6 is determined twice, for example the region between the determined positions at which the template is located may thus be used for further examination of the digital image 6. However, further suitable image analysis methods can be realised according to various exemplary embodiments.

In an exemplary embodiment the sign of skin ageing 7a, 7b determined and the image 6 may be compared with a series of images 8a, 8b stored in the memory 2, as is shown for example by a reference sign 10 in FIG. 2. In an exemplary embodiment the sign of skin ageing 7a, 7b determined in the image 6 is sent or stored or archived by the processor 3 in the memory 2 by a wireless connection 9. In another exemplary embodiment the processor 3 is connected to the memory 2 by a wired connection 9.

In an exemplary embodiment the system 200 comprises the features of the system 100 described in relation to FIG. 1.

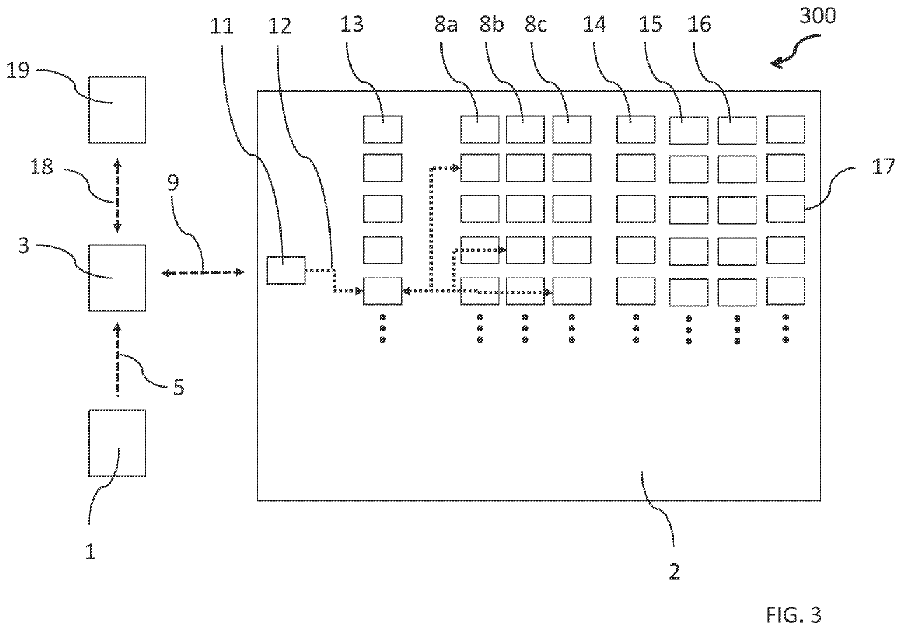

FIG. 3 shows a schematic depiction of a system 300 for determining an efficacy of a cosmetic treatment in accordance with an exemplary embodiment.

In an exemplary embodiment the system 300 comprise sensor 1 in the form of a camera for recording a digital image of a face of a user. The sensor 1 may be connected to the processor 3 via the wireless connection 5, for example a WLAN or Bluetooth connection. In various exemplary embodiments the processor 3 may be connected to a display device 19 by employing a wireless connection 18, for example a WLAN or Bluetooth connection.

In an exemplary embodiment the processor 3 is connected to the display device 19 by a wired connection 18. In an exemplary embodiment the system 300 comprises use of an app. For example the sensor 1 may thus be a digital camera of a smartphone or tablet, etc., and the processor 3 may be a processor of a smartphone, tablet, etc., and the display device 19 may be a touchscreen of a smartphone, of a tablet, etc. In an exemplary embodiment the sensor 1 is a camera in a smart mirror. The smart mirror may be provided as a display device 19. The user for example can transmit further personal information to the system 300 by employing an input via the smart mirror.

In an exemplary embodiment the processor 3 is integrated in a smartphone, in a tablet, or in a smart mirror. The display device 19 for example may be a touchscreen of the smartphone, of the tablet, or of the smart mirror. In an exemplary embodiment an information item may be transmitted from the processor 3 to the display device 19 by the connection 18. In another embodiment, an information item, for example, an input of an information item by the user via a touch-sensitive screen or touch screen of the display device 19, may be transmitted from the display device 19 to the processor 3 by the connection 18.

In an exemplary embodiment, by employing the display device 19, the user transmits additional information regarding his/her dietary habits, his/her general state of health, and further individual behavioural patterns to the processor 3 via the connection 18. The processor 3 may transmit the information input by the user to the memory 2 via the connection 9 and may store said information in the memory 2.

In an exemplary embodiment the processor 3 transmits the recorded digital image or digital image values via the connection 9 to the memory 2 for storing the digital image in a memory space 11 of the memory 2. In an exemplary embodiment the processor 3 examines the digital image stored or archived in the memory space 11 by employing digital image analysis and determines a sign of skin ageing or skin ageing information in the digital image. The determined skin ageing information item may be added for example to an image history 13 placed or generated in the memory 2 for the particular user. The particular skin ageing information items supplied to the image history 13 may be compared with a plurality of series of images 8a, 8b, 8c of a skin ageing information item or sign of skin ageing archived in the memory 2. A sign of skin ageing or a skin ageing information item may be, for example, a level of severity of a forehead line or a crow's foot, etc. A recommendation for use of certain care products can be given depending on the determined signs of skin ageing and a skin ageing severity.

In an exemplary embodiment the memory 2 comprises a target skin state information item 14. The target skin state information item 14 for example may comprise an information item that informs a particular user as to how far a treatment success has progressed or how far the particular user is behind a treatment success possible as a result of the treatment with a care product, based on a predefined treatment period, for example one week or two weeks, etc. In other words, a target skin state information item 14 for example may inform the user that the current skin ageing state which can be determined by employing the sensor 1 does not correspond to the skin ageing state stored in the memory 2 which for example can be achieved normally by other users of the same care product with the same treatment duration. The current treatment success consequently is compared, in an exemplary embodiment, with a corresponding target skin ageing state 14, in order to provide the user with feedback regarding success of a skin treatment. Furthermore, in an exemplary embodiment a recommendation for use of certain care products or care tips may be derived or provided to the user depending on the target skin ageing state 14. In an exemplary embodiment the target skin ageing state 14 is determined on the basis of literature data on the basis of other successful treatments of at least one user with a care product. In other words, a target skin ageing state 14 archived in the memory 2 may be associated with a predefined treatment time.

In an exemplary embodiment the memory 2 also comprises and/or stores a further information item 15, input for example by employing the display device 19, conducted via the connection 18 to the processor 3, and stored in the memory 2, regarding a dietary habit of the user, a general state of health of the user, and/or a further individual behavioural pattern of the user. A poor diet, for example the consumption of too much sugar, too much fatty food, gluten-rich foods, too many milk products, etc., may have unpleasant results for the health of the facial skin of a user. Thus, the facial skin, for example in the case of excess consumption of sugar, may lose its natural colour and its natural elasticity, may become thinner, and in particular lines may form in the eye region.

In an exemplary embodiment the memory 2 comprises a treatment success information item 16 of at least one further user. In an exemplary embodiment a treatment success of at least one user may be archived in the memory 2. In other words, for example with each detection of a skin ageing state of a user by employing the sensor 1, a currently measured skin ageing state or a treatment success can be stored in the memory 2 depending on a treatment period. Accordingly, a user may benefit from the stored skin ageing state of a user, the treatment period of which for example has finished, as a reference skin ageing state. Consequently, the user may be able to better monitor the individual efficacy of a cosmetic, which may lead to an increase in the motivation to carry out a longer-term treatment of the facial skin of the user.

In an exemplary embodiment the memory 2 also comprises a care product information item 17, for example information regarding a care product recommendation. The care product information item 17 for example may be information regarding a care product, for example a mask, a cream, a gel, etc. The care product information 17 for example may comprise an overview of the use of the particular care product or ingredients of the particular care product. Furthermore, the care product information 17 may provide for example information as to when and in what amount a care product should be applied to the skin.

In an exemplary embodiment the system 300 provides information regarding a current skin ageing state of a user by employing a skin ageing information item detected by employing the sensor 1, a skin ageing information item stored in the memory 2, a series of images of a plurality of series of images 8a, 8b, 8c, an archived target skin ageing state 14, a further information item 15, a treatment success information item 16, and/or a care product information item 17. A current skin ageing state of a user can be provided on the basis of just one of the aforementioned information items or states or on the basis of a plurality of the aforementioned information items or states.

In various exemplary embodiments the individual needs of the skin of the user may be investigated.

In various exemplary embodiments the system 300 comprises the features of system 200 described in relation to FIG. 2.

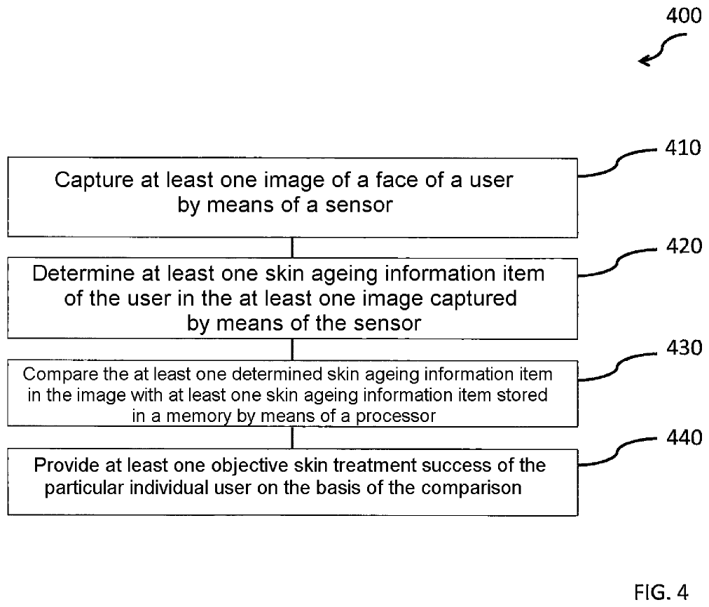

FIG. 4 schematically shows a method 400 for determining an efficacy of a cosmetic treatment in accordance with an exemplary embodiment.

In an exemplary embodiment the method 400 comprises capturing at least one image of a face of a user by employing a sensor (410), determining at least one skin ageing information item of the user in the at least one image (420) captured by employing the sensor, comparing the at least one determined skin ageing information item in the image with at least one skin ageing information item stored in a memory by employing a processor (430), and providing at least one objective skin treatment success of the particular individual user on the basis of the comparison (440).

In various exemplary embodiments the method 400 also comprises features which are described with reference to FIGS. 1, 2 and/or 3.

Various aspects of this disclosure will be presented hereinafter:

Exemplary embodiment 1 is a system for determining an efficacy of a cosmetic skin treatment. The system may comprise a sensor for determining a skin ageing information item of a user, a memory, wherein the memory is designed to store the skin ageing information of the user determined by employing the sensor; and a processor, wherein the processor is designed to determine an objective skin treatment success of the particular individual user on the basis of a comparison of a skin information item of the user measured by employing the sensor with a skin information item stored in the memory.

In exemplary embodiment 2 the subject matter of exemplary embodiment 1 may optionally include the fact that the sensor comprises a digital camera.

In exemplary embodiment 3 the subject matter of exemplary embodiment 1 may optionally include the fact that the sensor is integrated in a smartphone, in a tablet or in a smart mirror and/or the memory is integrated in the smartphone, in the tablet, in the smart mirror or in an external memory device, and/or the processor is integrated in the smartphone, in the tablet or in the smart mirror.

In exemplary embodiment 4 the subject matter of exemplary embodiment 1 may optionally include the fact that the memory also comprises a stored series of images of the sign of skin ageing, and that the series of images comprises various levels of severity of the sign of skin ageing.

In exemplary embodiment 5 the subject matter of exemplary embodiment 1 may optionally include the fact that the system is also designed to take into consideration a further individual information item, based on a dietary habit of the user, a state of health of the user, and/or an individual behavioural pattern of the user, when determining the objective skin treatment success of the user.

In exemplary embodiment 6 the subject matter of exemplary embodiment 1 may optionally include the fact that the processor is also designed to store the skin ageing information item for a user of a plurality of users of the system in the memory over the course of time.

In exemplary embodiment 7 the subject matter of exemplary embodiment 1 may optionally include the fact that the processor is also designed to compare an image recorded by employing the sensor with an image of a plurality of images stored in a series of images and/or to compare the image recorded by employing the sensor with an earlier skin ageing information item of the user stored in the memory.

In exemplary embodiment 8 the subject matter of exemplary embodiment 1 may optionally include the fact that the sensor also comprises a computer application (computer app).

In exemplary embodiment 9 the subject matter of exemplary embodiment 1 may optionally include the fact that the system is also designed to provide a skincare product tailored individually to the user and/or a skin recommendation tailored individually to the user on the basis of the comparison performed by employing the processor.

In exemplary embodiment 10 the subject matter of exemplary embodiment 9 may optionally include the fact that the system is also designed to determine an individual efficacy of a skin treatment of the user over the course of time on the basis of the care product provided to the user.

Exemplary embodiment 11 is a method for determining an efficacy of a cosmetic skin treatment. The method may comprise capturing an image of a face of a user by employing a sensor, determining a skin ageing information item of the user in the image captured by employing the sensor, comparing the determined skin ageing information item in the image with a skin ageing information item stored in a memory by employing a processor, and providing an objective skin treatment success of the particular individual user on the basis of the comparison.

In exemplary embodiment 12 the subject matter of exemplary embodiment 11 may optionally include the fact that the skin ageing information stored in the memory comprises a series of images of degrees of severity of a sign of skin ageing.

In exemplary embodiment 13 the subject matter of exemplary embodiment 11 may optionally include the fact that the comparison comprises a determination of a difference between the skin ageing information stored in the memory and a skin ageing information item measured later by the sensor.

In exemplary embodiment 14 the subject matter of exemplary embodiment 11 may optionally include the fact that the comparison comprises a determination of a difference between the skin ageing information measured by the sensor and a target skin ageing information item stored in the memory.

In exemplary embodiment 15 the subject matter of exemplary embodiment 11 may optionally include the fact that the method also comprises a provision of care recommendations and/or skincare products tailored individually to the skin ageing information on the basis of the skin ageing information measured by employing the sensor and/or a target skin ageing information item.

In exemplary embodiment 16 the subject matter of exemplary embodiment 11 may optionally include the fact that the comparison comprises a determination of a closest stored image from the series of images stored in the memory.

In exemplary embodiment 17 the subject matter of exemplary embodiment 11 may optionally include the fact that the determination also comprises a determination of a state of health, a dietary habit, and/or a further behavioural pattern of the user.

In exemplary embodiment 18 the subject matter of exemplary embodiment 11 may optionally include the fact that the comparison also comprises a comparison between a treatment success of the user and between a treatment success of a further user.

In exemplary embodiment 19 the subject matter of exemplary embodiment 11 may optionally include the fact that the capture of the image comprises a capture by employing a digital camera.

In exemplary embodiment 20 the subject matter of exemplary embodiment 11 may optionally include the fact that the method also comprises use of a computer application (computer app).

In exemplary embodiment 21 the subject matter of exemplary embodiment 11 may optionally include the fact that the method also comprises a provision of an objective estimation of an individual treatment success by employing a display device

In exemplary embodiment 22 the subject matter of exemplary embodiment 11 may optionally include the fact that the capturing of the image comprises a capturing of a digital image and the determination comprises an analysis of the digital image for a sign of skin ageing by employing a digital image analysis.

In exemplary embodiment 23 the subject matter of exemplary embodiment 11 may optionally include the fact that the skin ageing information comprises an information item regarding saggy jowls, nasolabial folds, forehead lines, crow's feet, bags under the eyes, wrinkles above the upper lip, age spots or a double chin.

In exemplary embodiment 24 the subject matter of exemplary embodiment 11 may optionally include the fact that the method also comprises a provision of an extent of a sign of skin ageing from a plurality of signs of skin ageing and/or a cumulated total value on the basis of the plurality of skin ageing information items.

In exemplary embodiment 25 the subject matter of exemplary embodiment 11 may optionally include the fact that the method also comprises a provision of a plurality of skin ageing information items for a user over the course of time.

In exemplary embodiment 26 the subject matter of exemplary embodiment 1 may optionally include the fact that the memory comprises a database of a server.

In exemplary embodiment 27 the subject matter of exemplary embodiment 1 may optionally include the fact that the memory is also designed to archive a plurality of skin ageing information items for a user over the course of time.

In exemplary embodiment 28 the subject matter of exemplary embodiment 1 may optionally include the fact that the system is also designed to archive a treatment success during a cosmetic treatment.

In exemplary embodiment 29 the subject matter of exemplary embodiment 1 may optionally include the fact that the system is also designed to determine an individual treatment success of a user on the basis of the treatment success of another user and/or literature data.

In exemplary embodiment 30 the subject matter of exemplary embodiment 1 may optionally include the fact that the system is also designed to provide the individual treatment success of the user graphically over the course of time on the basis of a plurality of determined measurement results of skin ageing states.

In exemplary embodiment 31 the subject matter of exemplary embodiment 1 may optionally include the fact that the system also comprises a display device, wherein the display device is designed to graphically display an individual skin treatment success and/or an individual skincare recommendation or an individual care product recommendation.

In exemplary embodiment 32 the subject matter of exemplary embodiments 9 or 31 may optionally be configured to place an online order for a skincare product provided to the user.

Further advantageous embodiments of the method will become clear from the description of the system, and vice versa.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A system for determining an efficacy of a cosmetic skin treatment, comprising:
a sensor configured to determine a skin ageing information item of a user;
a memory configured to store the skin ageing information item of the user determined by the sensor; and
a processor configured to determine an objective skin treatment success of the user on the basis of a comparison of the skin ageing information item of the user measured by the sensor with a stored skin ageing information item stored in the memory.

2. The system according to claim 1,
wherein the sensor comprises a digital camera.

3. The system according to claim 1,
wherein the sensor is integrated in a tablet or in a smart mirror, the memory is integrated in a smartphone, in a tablet, in a smart mirror or in an external memory device,
the processor is integrated in a smartphone, in a tablet, or in a smart mirror, or a combination thereof.

4. The system according to claim 1,
wherein the memory also comprises a stored series of images of the skin ageing information item of the user, and
wherein the series of images comprises different levels of severity of the skin ageing information item.

5. The system according to claim 1,
wherein the processor is configured to determine the objective skin treatment success of the user based on a further individual information item selected from a dietary habit of the user, a state of health of the user, a behavioral pattern of the user, or a combination thereof.

6. The system according to claim 1,
wherein the processor is also configured to store the skin ageing information item of the user of a plurality of users of the system in the memory over the course of time.

7. The system according to claim 1,
wherein the processor is also configured to compare an image recorded by the sensor with an image of a plurality of images stored in a series of images, to compare the image recorded by the sensor with an earlier skin ageing information item of the user stored in the memory, or both.

8. The system according to claim 1,
wherein the processor is configured to provide to the user a recommendation for a skincare product, a skin care recommendation or both on the basis of the comparison performed by the processor.

9. The system according to claim 8,
wherein the processor is configured to place an online order for the skincare product.

10. The system according to claim 8,
wherein the processor is configured to determine an efficacy of a skin treatment including the skincare product of the user over the course of time.

11. A method for determining an efficacy of a cosmetic skin treatment, the method comprising the steps of:
capturing an image of a face of a user by using a sensor;
determining a skin ageing information item of the user in the image captured by the sensor;
comparing the skin ageing information item in the image to a skin ageing information item stored in a memory by using a processor; and
providing an objective skin treatment success of the user on the basis of the comparison.

12. The method according to claim 11,
wherein the skin ageing information item stored in the memory comprises a series of images of levels of severity of a sign of skin ageing.

13. The method according to claim 11,
wherein the comparison comprises a determination of a difference between the skin ageing information item stored in the memory and the skin ageing information item captured by the sensor.

14. The method according to claim 11,
wherein the comparison comprises a determination of a difference between the skin ageing information item captured by the sensor and a target skin ageing information item stored in the memory.

15. The method according to claim 11,
further comprising recommending skin care, skincare products or both on the basis of the skin ageing information item captured by the sensor, the skin ageing information item stored in memory, or both.

16. A system for assessing skin ageing, the system comprising:
a camera configured to capture the image of a user;
a memory storing a first sign of skin ageing;
a processor configured to detect a second sign of skin ageing from the image of the user and compare the first sign of skin ageing to the second sign of skin ageing to provide to the user an assessment of the user's skin ageing.

17. The system of claim 16, wherein the first sign of skin ageing comprises a sign of skin ageing detected by the processor from a previous-captured image of the user.

18. The system of claim 16, wherein the camera is a camera of a smartphone, tablet, mobile phone, or a smart mirror.

19. The system of claim 16, wherein the first sign of skin ageing is one of a series of signs of skin ageing stored in the memory and the processor is configured to compare the second sign of skin ageing to the series of signs of skin ageing.

20. The system of claim 16, further comprising a display configured to display to the user the assessment of the user's skin ageing.
